(19) United States
(12) Patent Application Publication
(10) Pub. No.: US 2008/0014252 A1
(43) Pub. Date: Jan. 17, 2008

(54) TOPICAL COMPOSITIONS WITH LONG LASTING EFFECT

(76) Inventor: Keith DelPrete, Coopersburg, PA (US)

Correspondence Address: BLANK ROME LLP, 600 NEW HAMPSHIRE AVENUE, N.W., WASHINGTON, DC, 20037, US

(21) Appl. No.: 11/775,589

(22) Filed: Jul. 10, 2007

Related U.S. Application Data
(60) Provisional application No. 60/830,702, filed on Jul. 14, 2006.

(51) Int. Cl. A61K 31/135; A61K 31/075; A61K 31/4015; A61K 31/407; A61K 31/42; A61K 31/444; A61K 31/485; A61K 31/5415; A61K 31/58; A61K 31/60; A61K 31/603; A61K 33/04; A61K 33/06; A61K 33/24; A61K 33/30; A61K 33/32; A61K 33/34; A61K 38/02; A61K 38/16; A61K 9/70; A61P 21/02; A61P 25/04; A61P 25/24; A61P 31/04; A61P 31/10; A61P 35/00; A61P 9/10; A61P 9/12

(52) U.S. Cl. 424/449; 424/630; 424/639; 424/641; 424/646; 424/702; 424/722; 514/12; 514/165; 514/174; 514/2; 514/226.5; 514/282; 514/334; 514/378; 514/404; 514/413; 514/568; 514/653; 514/718; 514/789

(57) ABSTRACT

Topical pharmaceutical formulations and compositions for transdermal delivery of a variety of active agents are described. The formulations and compositions are formulated to provide a long lasting effect of the agent being delivered. The formulations and compositions of the present invention contain a transdermal vehicle and an adrenergic drug. The formulations and compositions may also contain guaifenesin.

### STATEMENT OF PRIORITY

This application claims priority to U.S. Provisional patent application Ser. No. 60/830,702 filed Jul. 14, 2006, which is hereby incorporated by reference in its entirety.

### FIELD OF THE INVENTION

The present invention relates to topical compositions for the transdermal delivery of active agents. More specifically, the present invention is a composition comprising an adrenergic drug and an active agent in a transdermal delivery vehicle, allowing for the active agents to be delivered directly to the tissue to be treated. The topical compositions of the invention allow the active agent to be delivered deep into the tissue and have a longer lasting effect than known topical compositions.

### BACKGROUND OF THE INVENTION

Transdermal delivery of active agents with topical compounds has distinct advantages over oral administration. With transdermal delivery, the active agent may be directly applied to the body part to be treated, helping to reduce side effects that may occur from the exposure of other body tissues or organs to the active agent that takes place during oral administration. Such side effects may include gastrointestinal discomfort and liver damage. Further, as transdermal compositions are applied directly to the affected area, they begin to take effect more quickly and allow for more control over the local concentration of the drug.

Topical treatments have previously been described in the art. U.S. Pat. Nos. 6,290,986, 6,572,880 and 6,479,074 to Murdock et al. describe compounds and methods for topical treatments for pain comprising psychopharmaceuticals, such as doxepin and lamotrigine in combination with muscle relaxants. U.S. Pat. No. 5,976,547 to Archer et al. describes therapeutic compounds for topical treatment of pain comprising an extract of the herb Arnica montana in combination with various other agents. These topical treatments have been used with various levels of effectiveness. Further, the topical treatments known in the art vary in their ease of use, as some compounds require that the patient apply them to the skin for up to 1 hour a day. There remains a need in the art for topical compounds that allow for the transdermal delivery of active agents that rapidly causes the alleviation of symptoms while maintaining relief for an extended period after application.

Muscle cramps occur in people of all ages and can disrupt daily activities and sleep. They occur most commonly in the elderly, with temporarily debilitating defects. Muscle cramps are also common among athletes and persons who perform a repetitive physical activity for long periods of time.

Currently, the most common treatment for muscle cramps is through preventative measures. Health care professionals typically recommend self-care measures to cramp sufferers, such as stretching exercises, good hydration practices and avoidance of repetitive strenuous activity. While these measures are helpful in lowering the incidence of cramps in a subject, they do not help with treatment of a cramp while it is actually occurring. Hence, there is a need in the art for medicaments that allow for the treatment and relief of cramps while they are occurring.

Fibromylagia is a chronic pain illness characterized by muscle aches, pain and stiffness. The frequent and sometimes debilitating pain of fibromyalgia disrupts the daily life and sleep patterns of sufferers of the disease, leading to severe fatigue and depression. Although the pain of fibromyalgia may be felt throughout the body, it is most common in the neck, back, shoulders, pelvic girdle and hands.

Fibromylagia affects approximately 3-6% of the population in the United States, and is much more common in women than in men. Current treatment of fibromyalgia may involve use of over-the-counter pain medications in composition with tricyclic antidepressants and scrotonin reuptake inhibitors to help relieve pain and improve sleep. Benzodiazepines, such as valium, may also be given to subjects that fail to respond to other medications. These treatments can help to reduce the painful symptoms of fibromyalgia, however, it often takes 30 minutes or more from the time the medication is taken until the pain begins to subside. Further, long term use of the above medications is usually not possible. Use of pain medications over a long term can cause the medication to become less effective over time. Long term use of oral pain medications is also not desirable as most of the organs of the body are exposed to the medication, where it can cause organ damage over time. Long term use of benzodiazepines is very dangerous due to the addictive properties of these medications. As such, there is a need in the art for fast acting long term pain relief that can be targeted to the site of pain.

### SUMMARY OF THE INVENTION

It is an object of the present invention to provide pharmaceutical formulations for topical application. The pharmaceutical formulations of the present invention contain an adrenergic drug and a transdermal vehicle that allows active agents in the formulation to penetrate the skin. The formulations are designated to penetrate deeply into skin and underlying tissues, giving a long lasting effect.

It is a further object of the invention to provide compositions for the topical treatment and prevention of ailments, including muscle pain and muscle cramps. The compositions of the invention contain an adrenergic drug and an active agent in a pharmaceutical vehicle suitable for transdermal delivery. The pharmaceutical vehicle may be a lecithin organogel or similar lecithin based transdermal delivery vehicle. The active agent may be chosen from a wide variety of agents, such as metals, metal salts, small molecule drugs and biotherapeutics. The compositions may also contain guaifenesin.

The compositions for treatment of ailments of the present invention act quickly upon application to the site of the ailment and provide long-lasting relief. The compositions of the present invention may be applied at the onset of symptoms of an ailment to treat the ailment and relieve the symptoms. The compositions of the present invention may also be applied to an area of a body where an ailment often occurs, preventing the onset of the ailment and/or its symptoms.

It is a further object of the invention to provide a method for the treatment or prevention of an ailment through topical application of a composition containing an adrenergic drug, an active agent and an transdermal vehicle. The method for treatment or prevention of an ailment can be used to treat a variety of ailments, depending on the active agent that is used in the composition. In the method of the present invention, the compositions may also contain guaifenesin.

### DETAILED DESCRIPTION OF THE INVENTION

The present invention is drawn to pharmaceutical formulations for topical application and transdermal delivery. The formulations include a transdermal vehicle capable of penetrating the skin and an adrenergic drug.

The present invention is also drawn to topical compositions for delivery of active agents that allow for a long lasting effect of the active agents. The compositions include a transdermal vehicle that allows for the delivery of an active agent through the skin to the muscle or other tissue to be treated and an adrenergic drug.

The formulations and compositions of the present invention preferably contain an adrenergic drug. In general, adrenergic drugs are compounds that stimulate the adrenergic nerves directly by mimicking the action of norepinephrine or indirectly by stimulating the release of norepinephrine. Non-limiting examples of adrenergic drugs contemplated by the invention include phenylephrine, epinephrine, norepinephrine, phenylpropanolamine, ephedrine, pseudoephedrine, and oxymetcoline. In preferred embodiments, the adrenergic drug is phenylephrine or epinephrine.

In preferred embodiments of the invention, the transdermal vehicle of the contains lecithin. As example of a transdermal vehicle contemplated for use in the present invention is lecithin organogel as described in U.S. Pat. No. 5,654,337 to Roentsch et al., which is hereby incorporated by reference herein. Lecithin organolgels are transdermal vehicles which are clear, thermostable, pharmaceutically acceptable gels that allow for the prolonged storage of active agents without loss in activity. It is also contemplated that other transdermal vehicles can be used, such as vesicular systems, lipid microspheres, lipid nanoparticles, lipid microemulsions, polymeric gels and dimethylsulfoxide (DMSO). It is of primary importance that the transdermal vehicle act to deliver the active agents through the skin to the tissue to be treated.

In preferred embodiments of the invention, the formulations and compositions also contain guaifenesin in addition to the transdermal vehicle and adrenergic drug. Guaifenesin (CAS 93-14-1) is a commercially available compound with the chemical name 3-(2-methoxyphenoxy)-1,2-propanediol and the chemical formula C₁₀H₁₄O₄. Guaifenesin may act as a rheological agent to thin out the compositions of the present invention, and hence, lower their viscosity.

In further embodiments of the invention, the compositions may also contain one or more active agents in addition to the transdermal vehicle and adrenergic drug. The active agents may be, metals or metal salts such as copper, magnesium, manganese, selenium, sodium, potassium, zinc, nickel, cobalt and iron, or the other metal salts thereof. They may be non-steroidal anti-inflammatory agents, such as Salicylates: Aspirin, Methyl salicylate, Diflunisal, Benorylate, Faislamine, Amoxiprin; Arylalkanoic acids: Diclofenac, Indomethacin, Sulindac; 2-Arylpropionic acids (profens): Carprofen, Fenoprofen, Flurbiprofen, Ibuprofen, Ketoprofen, Ketorolac, Loxoprofen, Naproxen, Tiaprofenic acid; N-Arylanthranilic acids (fenamic acids): Mefenamic acid, Meclofenamic acid; Pyrazolidine derivatives: Phenylbutazone, Oxyphenylbutazone; Oxicams: Piroxicam, Meloxicam; Coxibs: Celecoxib, Parecoxib, Etoricoxib; and Sulphonanilides: Nimesulide. They may be a topic anesthetic such as Benzocaine, Butamben, Dibucaine, Lidocaine, Menthol, Pramoxine and Tetracaine. They may be a steroidal anti-inflammatory, such as Betamethasone, Budesonide, Prednisone, Triamcinolone, Betamethasone, Cortisone, Dexamethasone, Hydrocortisone, Methylprednisolone and Prednisolone. The active agents may be antincoplastic agents such as Actinomycin, Dactinomycin, Anthracyclines, Doxorubicin, Daunorubicin, Epirubicin, Bleomycin, Plicamycin and mitomycin. They may be peptides, proteins, or hormones, such as platelet factors exhibiting angiostatic activity. They may be antineuralgic agents such as Capsaicin, Trazodone, Pregabalin, Maprotiline, Duloxetine, Hydantoin, Gabapentin and Carbamazepine. They may be muscle relaxants such as Cyclobenzaprine, Carisoprodol, Chlorphenesin, Chlorzoxazone, Metaxalone and Methocarbamol. They may be antifungal compounds such as Butoconazole, Clotrimazole, Econazole, Miconazole, Terconazole, Tioconazole, Fluconazole, Itraconazole and Ketoconazole. They may be anti-anginal compounds such as Amlodipine, Bepridil, Diltiazem, Felodipine, Flunarizine, Isradipine, Nicardipine, Nifedipine, Nimodipine and Verapamil. They may be cellulite reducers such as Theophylline, Aminophylline and Retinol (Vitamin A). They may be anti-depressants, such as Monoamine oxidase inhibitors (MAOI): Harmaline, Nialamide, Selegiline, Isocarboxazid, Iproniazid, Iproclozide, Phenelzine, Toloxatone, Tranylcypromine; Reversible Inhibitor of Monoamine oxidase A (RIMA): Brofaromine, Moclobemide; Dopamine Reuptake Inhibitor (DARI): Amineptine, Methylphenidate, Phenmetrazine, Vanoxerine, Nomifensine: Norepinephrine-dopamine reuptake inhibitors: Bupropion; Norepinephrine Reuptake Inhibitor (NRI) or (NARI): Atomoxetine, Reboxetine, Viloxazine, Maprotiline; Serotonin-Norepinephrine Reuptake Inhibitor (SNRI): Desipramine, Duloxetine, Milnacipran, Venlafaxine; Selective Serotonin Reuptake Inhibitor (SSRI): Alaproclate, Etoperidone, Citalopram, Escitalopram, Fluoxetine, Fluvoxamine, Paroxetine, Sertraline, Zimelidine; Selective Serotonin Reuptake Enhancer (SSRE): Tianeptine; Tricyclic antidepressants (TCA): Amitriptyline, Butriptyline, Clomipramine, Desipramine, Dibenzepin, Dothiepin, Doxepin, Imipramine, Lofepramine, Nortriptyline, Protriptyline, Trimipramine, Iprindole, Opipramol, Amoxapine: Tetracyclic antidepressants: Maprotiline, Mianserin, Trazodone, Nefazodone; and Noradrenergic and Specific Serotonergic Antidepressant (NaSSA): Mirtazapine. They may be antiepileptic agents such as Lamotrigine, Felbamate, Topiramate and Fosphenytoin. They may be opiates such as Tramadol, Morphine, Droperidol and DHEA. The above list is not meant to be inclusive and it should be apparent that there are additional agents contemplated for use with the present invention.

The active agents of the present invention may be presented in any form that allows them to be substantially active in the composition for topical application. Such forms may include, salts, free bases, acids and derivatives of the active agents. In the event of using a proteinaceous active compound, one must avoid adding the protein to a too-warm solution of solvent-polar lipid mixture as this might denature the protein if it is not thermostable. It is contemplated that more than more active agent may be present in the same composition.

The preferable concentrations of adrenergic drug in the compositions of the present invention is about 0.001% to about 0.1% of the mass of the final composition. When phenylephrine or epinephrine are present, it is most preferable that they are present at a concentration of about 0.005% to about 0.05% of the mass of the final composition. When present, the preferable concentration of guaifenesin in the compositions of the present invention is about 0.01% to about 30% of the mass of the final composition. More preferably, guaifenesin is present at about 8% to about 12% of the mass of the final composition. When present, the preferable concentration of active agents in the compounds of the present invention is about 10⁻¹⁰% to about 30% of the mass of the final composition. The preferable concentration for a specific active agent will vary based on the effectiveness and toxicity of the agent.

When the transdermal vehicle is a lecithin organogel, it may include the following ingredients in preferable concentrations of the mass of the final composition:

lecithin about 5% to about 30%;

surfactant—about 5% to about 30%;

urea about 5% to about 20%;

water—about 30% to about 60%.

Preferably, the surfactant of the transdermal vehicle is isopropyl myristate or isopropyl palmitate, which may both be present in the composition. However, other surfactants are contemplated by the invention, such as docusate sodium, Polysorbate 80, glycerin, polyethylene glycol, steatric acid, cetyl alcohol, stearyl alcohol and the like. These other surfactants may be used in combination or alone so that the final concentration of surfactant in the composition is about 5% to about 30% of the mass of the final composition.

The compositions of the present invention may include agents for adjusting the pH of the composition. When used, the preferred agent for adjusting pH is citric acid. It is also contemplated that the compositions of the present invention may contain other additives, such as fragrance and skin softeners such as aloe.

The compositions of the present invention may be prepared using compounding methods for transdermal compounds known in the art. In preferred methods, a transdermal vehicle, preferably lecithin, is dissolved in a surfactant at room temperature, possibly in the presence of an acid or base for pH regulation. Once the transdermal vehicle is completely dissolved in the surfactant, the mixture is combined with an emulsifier until a uniform, creamy texture is obtained. An adrenergic drug is then dissolved, along with any necessary salts or pH adjusting agents, in hot purified water (between 70 and 75° C.). While the adrenergic drug solution is still hot, it is added to the surfactant/emulsifier mixture and more surfactant is added if necessary. The entire mixture is mixed until a clear solution is formed. If desired, other compounds can be added to the composition at this point, such as guaifenesin or an active agent. This may be done by adding the compound either in a solid form or in a solution so that the compound has its desired concentration in the final solution. If the compound is added in a solution form, it is preferred that only a small amount of solution is added so that the composition maintains a gel-like consistency. Specific, non-limiting examples of forming certain embodiments of the compositions of the present invention are given in Examples 1 and 2 below.

For the treatment of an ailment, the compositions of the present invention are preferably applied at the onset of the ailment. Application typically involves use of a roll-on applicator, a spray bottle, a brush, a gauze, a tissue or the fingers. The compositions may be applied in a thin layer on the skin covering the area to be treated. The area may then be covered with a bandage or gauze, or may be left uncovered. It is further contemplated that the compositions of the present invention may be integrated directly into packaged bandages for convenient application. The bandages of the invention come with the compositions of the invention pre-applied to allow for easy administration to a subject.

After application of the compositions for treatment of an ailment, the active agents should being to relieve the ailment within about five minutes or less, or preferably, about two minutes or less, or most preferably, about one minute or less. After onset of relief, the effective relief of the ailment should continue for minutes, hours, or days. Effective relief as achieved through treatment means that the ailment is either eliminated completely or reduced in a manner that increases the comfort of the subject. As a non-limiting example, a composition of the present invention for treating muscle cramps may be applied at the time a cramp of the calf muscle is felt, providing relief of the cramp within one minute of application that lasts for approximately 12 hours.

For the prevention of an ailment, the compositions of the invention may be applied on a regular dosing schedule. The compositions should be applied in a manner analogous to that described for the treatment of an ailment, i.e. in a thin layer on the skin. For prevention of ailments, the compositions may be applied one or more times daily, one or more times weekly, or one or more times monthly. Prevention of an ailment includes prevention that completely eliminates the ailment as well as prevention that reduces the amount of discomfort that was felt while the ailment was untreated. As a non-limiting example, a composition may be applied to a subject's legs upon waking in the morning and just before going to sleep to help prevent leg cramps throughout the day and night.

The compositions of the present invention may require less frequent application as the course of treatment or prevention of an ailment continues. Because the compositions of the present invention are capable of delivering active agents deep into the tissue to be treated and allow for long lasting effects, residual active agent may remain in the tissue for an extended period, such as hours, days or weeks. The compositions may be applied as often as necessary or as recommended by a medical professional to treat or prevent the ailments described. It is of primary importance that the compositions are only applied with a frequency and in a manner that prevents the active agents from having a toxic effect on the subject. As a non-limiting example, a composition of the present invention may be applied twice daily for one week to prevent the onset of an ailment. The following week, it may be applied once daily to provide the same relief. The composition may then be subsequently applied every other day or even less frequently as the course of application continues.

For either treatment or prevention of ailments, the compositions of the present invention may be self-applied by the subject at home. They may also be applied by care givers and health care professionals either in a medical facility or elsewhere. For example, the compositions of the present invention may be applied by physical therapists before, during or after administering physical therapy.

The compositions of the present invention may be stored in a glass jar with a roll-on applicator. It is also contemplated that the compositions may be stored in other containers, including various types of jars and bottles made of glass, Plexiglas, plastic or other polymeric substance. In an alternative embodiment of the invention, the composition is stored in a spray bottle for application by spraying onto the skin.

In one embodiment of the present invention the compositions may be used to treat muscle cramps or other contractions. The terms cramps and contractions are meant to be used here as commonly known in the art. A cramp is typically a sudden and sharp muscle contraction or spasm that can be accompanied by pain. Cramps may last for a little as 10 seconds or less or for as much as 15 minutes or more. Contractions are characterized in that their onset may not be as sudden as that of a cramp, but may be more continuous or of longer duration. Contractions may or may not involve as sharp of a contraction or spasm in the muscle tissue and may or may not involve pain. During either a cramp or a contraction the muscle tissue may appear both visually and to the touch as a hard lump below the skin.

Embodiments of the compositions may be used to treat or prevent cramps and contractions of all kinds, including those of the legs, feet arms, hands back, abdomen, shoulders and neck. The cramps and contractions may be brought about by overuse of a muscle, such as during an athletic event, dehydration, fatigue, stretching, the menstrual cycle, or for no apparent reason. The cramps and contractions, especially those in the legs, may also be caused by medical reasons such as inadequate blood supply, the compression of nerves in the spine and the loss of potassium through the use of diuretic medications. It is also contemplated that the present invention may be used to treat or prevent cramps and contractions brought on by specific disorders, such as fibromyalgia.

The compositions of the present invention may also be used to treat or prevent pain, such pain caused by muscle cramps and contractions as described above. It is also contemplated that the compositions of the present invention may be used to treat other types of pain. The compositions may be used to treat other types of muscular pain, such as those caused by muscle strains, pulls and tears. They may also be used for other, non-muscular pains that can be treated topically, such as for arthritis and pains of the joints, tendons and ligaments.

The compositions of the present invention may also be used to treat other ailments. For example, the compositions of the present invention may be used to treat ailments such as angina, neoplasia, inflammation, cellulite, fungal infections, bacterial infections, depression, and high blood pressure. Preferably, these ailments will be treated using compositions of the present invention that contain active agents effective as treating the ailment. For example, a composition containing Amlodipine may be used to treat angina.

The embodiments and examples described herein are meant to give non-limiting examples of the compositions and methods of the present invention. It should be understood that there are other embodiments not specifically set forth above that fall within the spirit and scope of the invention as claimed.

### EXAMPLES

### Example 1

### Preparation of Copper Sulfate/Magnesium Sulfate/Phenylephrine Transdermal Composition

Stock Solutions

Copper Sulfate 4x

1. 1 g Copper sulfate (Cuprum metallicum 6X H.P.U.S.) was dissolved in 10 ml purified water to form a mother solution.

2. 1 ml of the solution of step #1 was taken and diluted in 10 ml purified water.

3. 1 ml of the solution of step #2 was taken and diluted 10 ml purified water.

4. 1 ml of the solution of step #3 was taken and diluted 10 ml purified water.

5. 1 ml of the solution of step #4 was taken and diluted 10 ml purified water.

6. 1 ml of the solution of step #5 was taken and diluted 10 ml purified water.

7. 1 ml of the solution of step #6 was taken and diluted 10 ml purified water. This 6× solution was used in the final formulation below.

Magnesium Sulfate 3x

1. 1 g of Magnesium sulfate (6X H.P.U.S.) was dissolved in 10 ml purified water to form a mother solution.

2. 1 ml of the solution of step #1 was taken and diluted 10 ml purified water.

3. 1 ml for the solution of step #2 was taken and diluted 10 ml purified water.

4. 1 ml for the solution of step #3 was taken and diluted 10 ml purified water.

5. 1 ml of the solution of step #4 was taken and diluted 10 ml purified water.

6. 1 ml of the solution of step #5 was taken and diluted 10 ml purified water.

7. 1 ml of the solution of step #6 was taken and diluted 10 ml purified water. This 6× dilution was used in the final formulation below.

Lecithin/Isopropyl Palmitate Stock Solution

| | | |
|---|---|---|
| Lecithin/isopropyl Palmitate solution | 220 | ml |
| Lecithin Soya Granular | 100 | g |
| Isopropyl Palmitate, NF, Cosmetic grade | 117 | ml |
| Sorbic Acid, NF, Cosmetic grade | 0.66 | g |

Lecithin Soya Granular and Sorbic Acid were dispersed in Ispopropyl Palmitate and allowed to stand at room temperature until all particles were dissolved and a clear product remained.

| | 100 ml Total | |
|---|---|---|
| FORMULA: | Volume | |
| Copper Sulfate step #7 solution | 0.05 | ml |
| Magnesium Sulfate step #7 solution | 0.05 | ml |
| Guaifenesin 10% | 10 | g |
| Phenylepherine HCl | 10 | mg |
| Citric Acid | 2.5 | g |
| Urea USP | 10 | g |
| Docusate Sodium USP 85% (15% sodium benzoate) | 10 | g |
| Lecithin/Isopropyl Palmitate stock solution | 22 | ml |
| Polysorbate 80 | 10 | g |
| Isopropyl Palmitate NF | 2.8 | ml |
| Purified water | 45 | ml |

1. The Urea, Citric acid, Phenylepherine HCl, and Guaifenesin were dissolved in 45 ml of hot (70 to 75° C.) purified water.

2. The Copper sulfate and Magnesium sulfate dilutions were added to step 1 and the resultant solution was mixed.

3. Polysorbate 80. Docusate sodium, and Lecithin/Isopropyl palmitate solution were mixed until a creamy and uniform mixture formed.

4. While the mixture of steps 1 and 2 was still hot, it was added to step 3, followed by addition of 2.8 ml isopropyl palmitate. The resultant solution was mixed until a clear amber solution was formed.

### Example 2

### Preparation of Phenylephrine Transdermal Gel Base

Step 1: Lecithin/Isopropyl Palmitate Solution

| | 220 mL | |
|---|---|---|
| Lecithin Soya Granular | 100 | g |
| Isopropyl Palmitate, NF, Cosmetic Grade | 117 | mL |
| Sorbic Acid, NF, Cosmetic Grade | 0.66 | g |

The lecithin soya granular and sorbic acid were dispersed in isopropyl palmitate and allowed to stand at room temperature until all particles were dissolved and a clear product was formed.

Step 2: Gel Base

| | 100 g | |
|---|---|---|
| Polysorbate 80 NF | 10 | g |
| Lecithin/Isopropyl Palmitate Solution (from Step 1) | 22 | mL |
| Docusate Sodium, USP 85% (15% Sodium Benzoate) | 10 | g |
| Urea, USP | 10 | g |
| Phenylephrine Hydrochloride, USP | 10 | mg |
| Purified Water, USP | 45 | mL |
| Citric Acid, USP Hydrous Powder | 2.5 | g |
| Isopropyl Myristate NF or Isopropyl Palmitate NF | 2.8 | mL |

A. The Polysorbate 80, docusate sodium and lecithin/isopropyl palmitate solution were mixed until a creamy and uniform texture was achieved.

B. The urea, citric acid and phenylephrine hydrochloride were dissolved in 45 mL of hot (70 to 75 degree C.) purified water.

C. While the solution from step B is still hot, it was added to the mixture from step A, followed by the addition of 2.8 mL of isopropyl palmitate. The entire mixture was mixed until a clear amber solution formed.

Step 3: Guaifenesin 10% Topical Solution

| | 100 g | |
|---|---|---|
| Guaifenesin USP | 10 | g |
| Gel base (from Step 2) | q.s. 100 | g |

A. Guaifenesin was weighed out and placed in a mortar to reduce particle size with titration.

B. Enough gel base (from Step 2) was added to bring to a final weight of 100 g. followed by mixing until a clear yellow solution was formed.

### Example 3

### Preparation of Epinephrine Transdermal Gel Base

Step 1: Lecithin/Isopropyl Palmitate Solution

| | 220 mL | |
|---|---|---|
| Lecithin Soya Granular | 100 | g |
| Isopropyl Palmitate, NF, Cosmetic Grade | 117 | mL |
| Sorbic Acid, NF, Cosmetic Grade | 0.66 | g |

The lecithin soya granular and sorbic acid were dispersed in isopropyl palmitate and allowed to stand at room temperature until all parties were dissolved and a clear product formed.

Step 2: Gel Base

| | 100 | g |
|---|---|---|
| Polysorbate 80 NF | 10 | g |
| Lecithin/Isopropyl Palmitate Solution (from Step 1) | 22 | mL |
| Docusate Sodium, USP 85% (15% Sodium Benzoate) | 10 | g |
| Urea, USP | 10 | g |
| Epinephrine Hydrochloride, USP | 50 | mg |
| Purified Water, USP | 45 | mL |
| Citric Acid, USP Hydrous Powder | 2.5 | g |
| Isopropyl Myristate NF or Isopropyl Palmitate NF | 2.8 | mL |

A. The Polysorbate 80, docusate sodium and lecithin/isopropyl palmitate solution were mixed until a creamy and uniform texture was achieved.

B. The urea, citric acid and epinephrine hydrochloric were dissolved in 45 mL of hot (70 to 75 degree C.) purified water.

C. While the solution from Step B was still hot, it was added to the mixture from step A, followed by the addition of 2.8 mL of isopropyl palmitate. The entire mixture was mixed until a clear amber solution formed.

Step 3: Guaifenesin 10% Topical Solution

| | 100 g | |
|---|---|---|
| Guaifenesin USP | 10 | g |
| Gel base (from Step 2) | q.s. 100 | g |

A. Guaifenesin was weighed out and placed in a mortar to reduce particle size with titration.

B. Enough gel base (from step 2) was added to bring to a final weight of 100 g, followed by mixing until a clear yellow solution formed.

### Example 4

### Treatment of Lower Back Spasms

A 54 year old women suffered from severe lower back spasms due to and accident for 10 years. Before treatment, her pain was described as an 8 on a scale from 1 to 10, with 10 being the most severe. Previously, she had been treated with oral medication for pain and with muscle relaxants without significant benefit. After treatment with other topical pain relievers, her pain diminished to a level 2, but the duration of pain relief was approximately 2 hours. After the composition of Example 1 was used for treatment, the duration of pain relief increased to 6 to 8 hours. She reported no skin irritation or other adverse affects.

### Example 5

### Treatment of Leg Cramps

A 56 year old women suffered from chronic leg cramps nightly for 7 years without relief from other methods. After treatment with the composition of Example 1, she reported relief of symptom in 1 to 2 minutes after application. She later applied the product at bedtime to prevent the onset of cramps. This proved to be successful. She reported no skin irritation or other adverse affects.

What is claimed is:

1. A pharmaceutical formulation for topical application for transdermal delivery of an active agent, comprising:
a transdermal vehicle; and
an adrenergic drug;
in amounts sufficient for effective transdermal delivery of the active agent.

2. The formulation of claim 1, wherein the adrenergic drug is selected from the group consisting of: phenylephrine, epinephrine, norepinephrine, phenylpropanolamine, ephedrine, pseudoephedrine and oxymetcoline.

3. The formulation of claim 2, wherein the adrenergic drug is phenylephrine.

4. The formulation of claim 2, wherein the adrenergic drug is epinephrine.

5. The formulation of claim 1, further comprising guaifenesin.

6. A pharmaceutical composition for topical application for treatment or prevention of an ailment comprising,
a transdermal vehicle;
an adrenergic drug; and
an active agent;
in amounts effective to treat or prevent the ailment.

7. The composition of claim 6, further comprising guaifenesin.

8. The composition of claim 6, wherein the active agent is selected from the group consisting of: metals, metal salts, non-steroidal anti-inflammatory agents, steroidal anti-inflammatory agents, antincoplastic agents, peptides, proteins, hormones, antineuralgic agents, muscle relaxants, antifungal compounds, anti-anginal compounds, cellulite reducers, anti-depressants, antiepileptic agents and opiates.

9. The composition of claim 6, wherein the active agent is selected from the group consisting of: copper, magnesium, manganese, selenium, sodium, potassium zinc, nickel, cobalt, iron, or salts thereof.

10. The composition of claim 6, wherein the active agent is selected from the group consisting of: Aspirin, Methyl salicylate, Diflunisal, Benorylate, Faislamine, Amoxiprin, Diclofenac, Indomethacin, Sulindac, Carprofen, Fenoprofen, Flurbiprofen, Ibuprofen, Ketoprofen, Ketorolac, Loxoprofen, Naproxen, Tiaprofenic acid, Mefenamic acid, Meclofenamic acid, Phenylbutazone, Oxyphenylbutazone, Piroxicam, Meloxicam, Celecoxib, Parecoxib, Etoricoxib, Nimesulide, Benzocaine, Butamben, Dibucaine, Lidocaine, Menthol, Pramoxine, Tetracaine, Betamethasone, Budesonide, Prednisone, Triamcinolone, Betamethasone, Cortisone, Dexamethasone, Hydrocortisone, Methylprednisolone, Prednisolone, Actinomycin, Dactinomycin, Doxorubicin, Daunorubicin, Epirubicin, Bleomycin, Plicamycin, Mitomycin, Capsaicin, Trazodone, Pregabalin, Maprotiline, Duloxetine, Hydantoin, Gabapentin, Carbamazepine, Guafenesin, Cyclobenzaprine, Carisoprodol, Chlorphenesin, Chlorzoxazone, Metaxalone, Methocarbamol, Butoconazole, Clotrimazole, Econazole, Miconazole, Terconazole, Tioconazole, Fluconazole, Itraconazole, Ketoconazole, Amlodipine, Bepridil, Diltiazem, Felodipine, Flunarizine, Isradipine, Nicardipine, Nifedipine, Nimodipine Verapamil, Theophylline, Aminophylline Retinol (Vitamin A), Harmaline, Nialamide, Selegiline, Isocarboxazid, Iproniazid, Iproclozide, Phenelzine, Toloxatone, Tranylcypromine, Brofaromine, Moclobemide, Amineptine, Methylphenidate, Phenmetrazine, Vanoxerine, Nomifensine, Bupropion, Atomoxetine, Reboxetine, Viloxazine, Maprotiline, Desipramine, Duloxetine Milnacipran, Venlafaxine, Alaproclate, Etoperidone, Citalopram, Escitalopram, Fluoxetine, Fluvoxamine, Paroxetine, Sertraline, Zimelidine, Tianeptine, Amitriptyline, Butriptyline, Clomipramine, Desipramine, Dibenzepin, Dothiepin, Doxepin, Imipramine, Lofepramine, Nortriptyline, Protriptyline, Trimipramine, Iprindole, Opipramol, Amoxapine, Maprotiline, Mianserin, Trazodone, Nefazodone, Mirtazapine, Lamotrigine, Felbamate, Topiramate, Fosphenytoin, Tramadol, Morphine, Droperidol and DHEA.

11. The composition of claim 6, wherein the adrenergic drug is present at a concentration of about 0.001 to about 10% of the mass of the final composition.

12. The composition of claim 6, wherein the adrenergic drug is present at a concentration of about 0.005 to about 0.05% of the mass of the final composition.

13. The composition of claim 6, wherein the active agent is present at a concentration of about 10⁻¹⁰ to about 30% of the mass of the final composition.

14. The composition of claim 7, wherein the guaifenesin is present at a concentration of about 0.01 to about 30% of the mass of the final composition.

15. The composition of claim 7, wherein the guaifenesin is present at a concentration of about 8 to about 12% of the mass of the final composition.

16. The composition of claim 6, wherein the transdermal vehicle is a lecithin organogel.

17. The composition of claim 6, wherein the composition comprises as a percentage of the mass of the final composition:
lecithin—about 5% to about 30%;
a surfactant—about 5% to about 30%;
urea—about 5% to about 20%;
water about 30% to about 60%.

18. The composition of claim 17, wherein the surfactant is selected from the group consisting of: isopropyl myristate, isopropyl palmitate, docusate sodium, Polysorbate 80, glycerin, polyethylene glycol, steatric acid, cetyl alcohol, stearyl alcohol and mixtures thereof.

19. The composition of claim 17, wherein the surfactant is isopropyl myristate.

20. The composition of claim 17, wherein the surfactant is isopropyl palmitate.

21. A method for the topical treatment or prevention of an ailment comprising;
administering to a part of the body of a subject in need of treatment or prevention of an ailment a composition comprising:
a transdermal vehicle;
an adrenergic drug; and
an active agent;
in amounts effective to treat or prevent the ailment.

22. The method of treatment or prevention of claim 21, wherein the adrenergic drug is phenylephrine.

23. The method of treatment or prevention of claim 21, wherein the adrenergic drug is epinephrine.

24. The method of treatment or prevention of claim 21, wherein the transdermal vehicle is a lecithin organogel.

25. The method of treatment or prevention of claim 21, wherein the composition further comprises guafenesin.

26. The method of treatment or prevention of claim 21, wherein the active agent is selected from the group consisting of: metals, metal salts, non-steroidal anti-inflammatory agents, steroidal anti-inflammatory agents, antincoplastic agents, peptides, proteins, hormones antineuralgic agents, muscle relaxants, antifungal compounds, anti-anginal compounds, cellulite reducers, anti-depressants, antiepileptic agents and opiates.

27. The method of treatment or prevention of claim 21, wherein the active agent is selected from the group consisting of: copper, magnesium, manganese, selenium, sodium, potassium zinc, nickel, cobalt, iron, or salts thereof.

28. The method of treatment or prevention of claim 21, wherein the active agent is selected from the group consisting of: Aspirin, Methyl salicylate, Diflunisal, Benorylate, Faislamine, Amoxiprin, Diclofenac, Indomethacin, Sulindac, Carprofen, Fenoprofen, Flurbiprofen, Ibuprofen, Ketoprofen, Ketorolac, Loxoprofen, Naproxen, Tiaprofenic acid, Mefenamic acid, Meclofenamic acid, Phenylbutazone, Oxyphenylbutazone, Piroxicam, Meloxicam, Celecoxib, Parecoxib, Etoricoxib, Nimesulide, Benzocaine, Butamben, Dibucaine, Lidocaine, Menthol, Pramoxine Tetracaine, Betamethasone, Budesonide, Prednisone, Triamcinolone, Betamethasone, Cortisone, Dexamethasone, Hydrocortisone, Methylprednisolone, Prednisolone, Actinomycin, Dactinomycin, Doxorubicin, Daunorubicin, Epirubicin, Bleomycin, Plicamycin, Mitomycin, Capsaicin, Trazodone, Pregabalin, Maprotiline, Duloxetine, Hydantoin, Gabapentin, Carbamazepine, Cyclobenzaprine, Carisoprodol, Chlorphenesin, Chlorzoxazone, Metaxalone, Methocarbamol, Butoconazole, Clotrimazole, Econazole, Miconazole, Terconazole, Tioconazole, Fluconazole, Itraconazole, Ketoconazole, Amlodipine, Bepridil, Diltiazem, Felodipine, Flunarizine, Isradipine, Nicardipine, Nifedipine, Nimodipine Verapamil, Theophylline, Aminophylline Retinol (Vitamin A), Harmaline, Nialamide, Selegiline, Isocarboxazid, Iproniazid, Iproclozide, Phenelzine, Toloxatone, Tranylcypromine, Brofaromine, Moclobemide, Amineptine, Methylphenidate, Phenmetrazine, Vanoxerine, Nomifensine, Bupropion, Atomoxetine, Reboxetine, Viloxazine, Maprotiline, Desipramine, Duloxetine, Milnacipran, Venlafaxine, Alaproclate, Etoperidone, Citalopram, Escitalopram, Fluoxetine, Fluvoxamine, Paroxetine, Sertraline, Zimelidine, Tianeptine, Amitriptyline, Butriptyline, Clomipramine, Desipramine, Dibenzepin, Dothiepin, Doxepin, Imipramine, Lofepramine, Nortriptyline, Protriptyline, Trimipramine, Iprindole, Opipramol, Amoxapine, Maprotiline, Mianserin, Trazodone, Nefazodone, Mirtazapine, Lamotrigine, Felbamate, Topiramate Fosphenytoin, Tramadol, Morphine, Droperidol and DHEA.

29. The method of treatment or prevention of claim 21, wherein the ailment is a muscle cramp.

30. The method of treatment or prevention of claim 21, wherein the ailment is pain.

31. The method of treatment or prevention of claim 30, wherein the pain is muscle pain.

32. The method of treatment or prevention of claim 30, wherein the pain is joint pain.

33. The method of treatment or prevention of claim 21, wherein the ailment is fibromylagia.

34. The method of treatment or prevention of claim 21, wherein the ailment is angina.

35. The method of treatment or prevention of claim 21, wherein the ailment is neoplasia.

36. The method of treatment or prevention of claim 21, wherein the ailment is inflammation.

37. The method of treatment or prevention of claim 21, wherein the ailment is cellulite.

38. The method of treatment or prevention of claim 21, wherein the ailment is a fungal infection.

39. The method of treatment or prevention of claim 21, wherein the ailment is a bacterial infection.

40. The method of treatment or prevention of claim 21, wherein the ailment is depression.

41. The method of treatment or prevention of claim 21, wherein the ailment is high blood pressure.

42. The method of treatment or prevention of claim 21, wherein said treating or preventing begins to occur within 5 minutes of the application of the composition.

43. The method of treatment or prevention of claim 21, wherein said treating or preventing beings to occur within 2 minutes of the application of the composition.

44. The method of treatment or prevention of claim 21, wherein said treating or preventing beings to occur within 1 minute of the application of the composition.

45. The method of treatment of claim 21, wherein said treating or preventing effectively lasts for at least 12 hours.

46. The method of treatment of claim 21, wherein said treating or preventing effectively lasts for at least 24 hours.

47. The method of treatment of claim 21, wherein the frequency of said administering decreases with the course of treatment.

48. A bandage for the topical treatment of an ailment comprising the composition of claim 6.

49. A gauze for the topical treatment of an ailment comprising the composition of claim 6.
